(19) United States
(12) Patent Application Publication
(10) Pub. No.: US 2013/0102020 A1
(43) Pub. Date: Apr. 25, 2013

(54) Detection and Characterization of Protein Interactions, Protein Interaction Modulation and Protein Interaction Modulators

(71) Applicants: Ray Clifford Perkins, (US); Sarah-Ellen Perkins Leonard, (US); Paul Johann Andre Kenis, (US)

(72) Inventors: Ray Clifford Perkins, (US); Sarah-Ellen Perkins Leonard, (US); Paul Johann Andre Kenis, (US)

(21) Appl. No.: 13/277,748

(22) Filed: Oct. 20, 2011

(51) Int. Cl. G01N 33/68; G01N 27/72; C12Q 1/02

(52) U.S. Cl. 435/29; 436/86

(57) ABSTRACT

Protein interactions, protein interaction modulation, and protein interaction modulators can be detected and characterized through assessment of differential angular mobility and/or differential polarity exhibited by protein interaction reactants and products.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

Proteins are engaged in hundreds of millions, if not billions, of interactions with other proteins, DNA, RNA, cells and cell membranes. These interactions control many biological processes, including those that represent the molecular basis of many diseases. Rapid, reliable determination of protein interactions in bio-relevant media and interaction modulation by small molecules and drugs has the potential to have a substantial impact on many areas of biology and biomedicine. Methodical detection and characterization of protein interactions would also benefit the development of biomarkers, the diagnosis of disease, and the monitoring of medical treatment efficacy. No general methodology or combination of methodologies exists to detect and characterize protein interactions.

Individual proteins participate in multiple interactions with diverse binding partners. Interactions may include formation of covalent bonds, but the majority of protein interactions exist in dynamic equilibria. Each of these equilibria may shift depending on modest changes in conditions or the presence or absence of a chemical, biochemical or biological entity. Methods that include separation of media components, e.g. a wash or precipitation step, disturb dynamic equilibria and potentially distort, disguise or even eliminate interactions, particularly “weak” interactions. Included among such methods are complex immunoprecipitation (Co-IP), protein microarrays, most protein affinity assays and surface plasmon resonance (SPR).

Proteins are large molecules on a molecular scale. Methods that include attachment of one potential binding partner to a solid surface, regardless of that surface's nature or composition, preclude large-molecule approach in the vicinity of the attachment site. Such methods include protein microarrays, most protein affinity assays and SPR.

Protein interaction partners vary in kind and include other proteins, polynucleotides and extended structures such as cells and cell membranes. While most extant methods accommodate a subset of potential inter-actors, none accommodate the entire list. For example, none of the hybridized protein methods, e.g. Yeast Two-Hybrid (YTH), can assess the binding of a protein to an intact leukocyte. The same shortcoming exists for Co-IP, SPR, mass spectrometry (MS) and the balance of all existing methods.

Non-covalent protein associations may reflect electro-dynamic or lipophilic interactions. The conditions required for execution of MS discriminate against lipophilic interactions, providing a false negative for this entire segment of interactions.

Protein interactions are subject to modulation by many factors: temperature, pH, ionic strength and the presence or absence of chemical, biochemical and biological entities. Any physiologically relevant assessment of protein interactions must accommodate interaction detection under all interaction modulation conditions. All existing methods appear to fail in this regard. As an obvious example, a test pharmaceutical may be toxic to yeast precluding the use of YTH.

All interacting partners experience a change in mobility, both translational and angular. This invention exploits changes in angular mobility induced by protein interactions. Of extant methods sensitive to changes in angular mobility, electron magnetic resonance (EMR, also known as Electron Spin Resonance (ESR) or Electron Paramagnetic Resonance (EPR)) and fluorescence polarization (FP) spectroscopies dominate. As tools for detection of changes in angular mobility, both EMR and FP generally employ reporters, or labels, spin labels in the case of EMR and fluorophores in the case of FP. While the combination of EMR and spin labeling (SL) covers a uniquely broad range of angular mobility rates, fluorophores are more restricted in range. Therefore, of the two, EMR and SL is the method of choice for this invention.

By its very nature SL reports on the angular mobility of a restricted region of the labeled protein, reflecting the angular mobility of a single amino acid. The angular mobility of a single amino acid is comprised of multiple, dynamic contributors: the overall mobility of the protein as an equivalent solid body or “global mobility”, and “local mobility” relative to the backbone of the protein itself. These variable contributors to “site mobility” are a key area of concern in demonstrating the general utility of methods in this invention. Research has demonstrated, for example, that site mobility varies considerably from amino acid to amino acid within a protein. Sites whose mobility approximate that of global mobility are those (or near those) that maintain the secondary (and tertiary) structure of the protein. Other sites exhibit substantial local mobility.

In a limited number of studies, EMR and SL have been successfully employed in protein interaction studies for which the interaction pairs were known from prior work: e.g. actin/myosin and sickle cell hemoglobin. The objective of such studies was characterization of the interaction complex itself, e.g. determination of the relative orientations of the interacting partners. A pre-condition of such studies was label site mobility equivalence to global mobility. As such an equivalence cannot be assumed a priori, given the variability in site mobility within a given protein, prior lines of research cannot and do not anticipate the general utility of EMR and SL in detection of protein interactions. In contrast, this invention imposes none of the constraints of earlier work and demonstrates the general utility of EMR and SL for detection and characterization of protein interactions, regardless of label site selection. It is also noted that the EMR response of spin labels is dependent on label site polarity as well as label site mobility. Variations in site polarity that may accompany protein interactions also contribute to the detection of those interactions.

### SUMMARY OF THE INVENTION

This invention demonstrates the utility of electron magnetic resonance (EMR, also known as Electron Spin Resonance (ESR) or Electron Paramagnetic Resonance (EPR)) and spin labeling (SL) as a general purpose approach in the detection of protein interactions, protein interaction modulation, and protein interaction modulators. The prime determinant of binding is differential angular mobility of the protein's labeled site, regardless of the site's mobility relative to that of the protein as a whole. The approach is both qualitative in its discernment of binding and quantitative sufficient for calculation of association constants. Modulation of the interaction by any change in conditions or media is readily apparent and chemical, biochemical or biological modulators readily detected and characterized. Methods require no component separation and may be performed in media of any bio-relevant complexity.

### BRIEF DESCRIPTION OF THE DRAWINGS

Drawing 1 of 6 shows the partial conversion of phosphorylase B into phosphorylase A;

Drawing 2 of 6 shows the low-field 40% of the spectra in Drawing 1 and digital titration isolating phosphorylase A content;

Drawing 3 of 6 shows the binding of protease to whole-protein substrate during active proteolysis;

Drawing 4 of 6 shows inhibition of protease binding to whole-protein substrate;

Drawings 5 of 6 & 6 of 6 show binding of protease inhibitor to multiple proteases.

### DETAILED DESCRIPTION OF THE INVENTION

This invention utilizes Electron Magnetic Resonance (EMR, also known as Electron Spin Resonance (ESR) or Electron Paramagnetic Resonance (EPR)) spectroscopy and spin labeling (SL) to detect and characterize protein interactions. At the heart of the invention is the ability to discern protein interactions from a combination of changes in angular mobility and/or polarity of the labeled site concurrent with the interaction event. Implementation of spin labeling in all its embodiments including site-directed spin labeling is well known and entirely analogous to other labeling methods such as fluorescent labeling. The combination of EMR in all its modes of operation and SL provide angular mobility sensitivity for correlation times from a tenth of a picosecond to a tenth of a second, corresponding to Stokes-Einstein-Debye molecular weights of 100 to 1,000,000 Daltons respectively. Methods included in this invention are both qualitative and quantitative, demonstrating both the fact and extent of interaction. Measurements are performed in real time, do not rely on media separation, and can be performed in media of any desired complexity. This invention applies to but is not limited to protein interactions with other proteins, polynucleotides and extended structures such as cells and cell membranes. Both “strong” and “weak” interactions are detectable and subject to quantitative characterization. Label site selection is infinitely flexible permitting internal confirmation of results and insurance of relevant bio-activity. Demonstrated herein are examples of the invention that anticipate the more challenging interaction situations to be encountered.

The ability to characterize protein interactions per se encompasses, through direct comparison of data, protein interaction modulation (“modulation”) regardless of the modulation mechanism. Chemical, biochemical or biological entities that act as protein interaction modulators (“modulators”) are readily identified and their actions characterized. Modulator interactions with interaction participants or any other chemical, biochemical or biological entity may also be assessed.

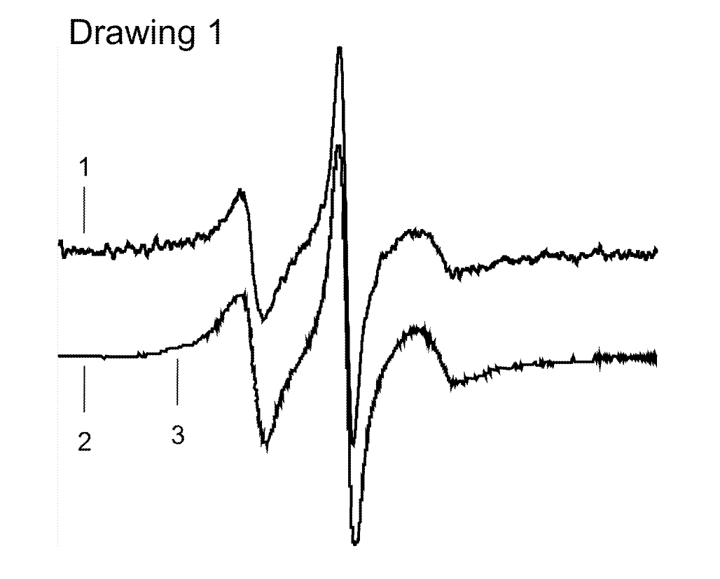

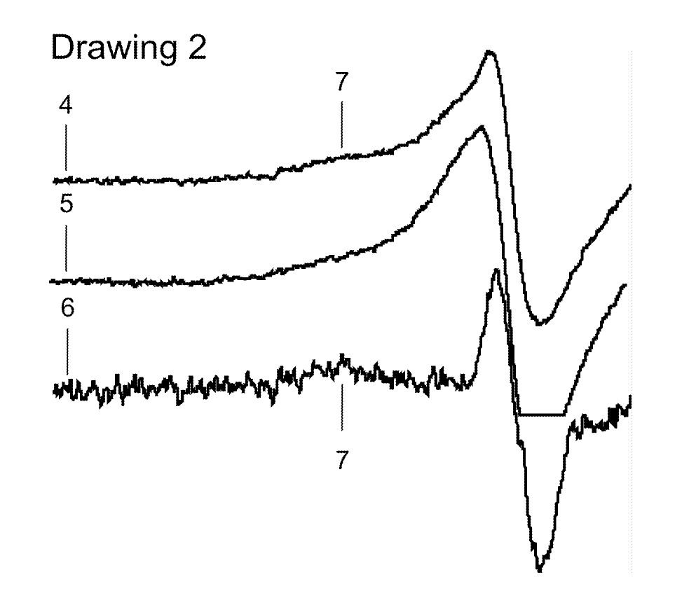

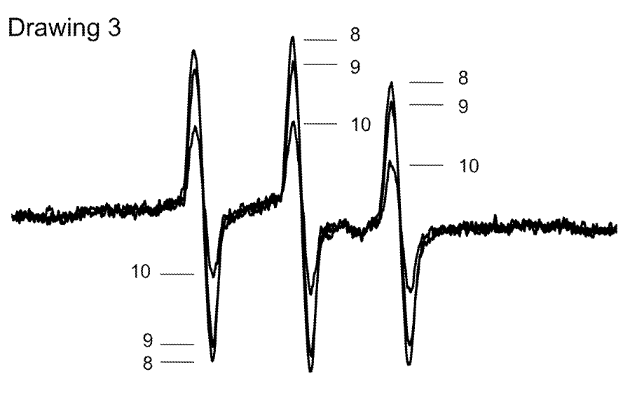

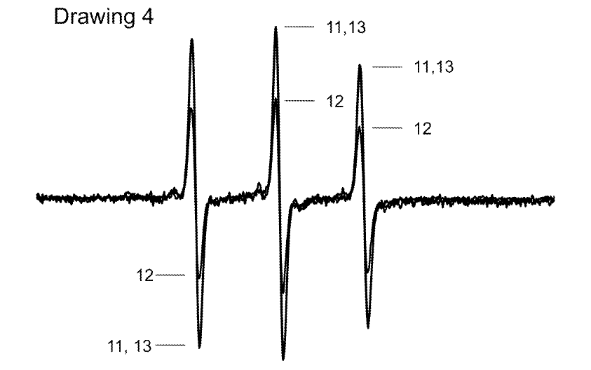

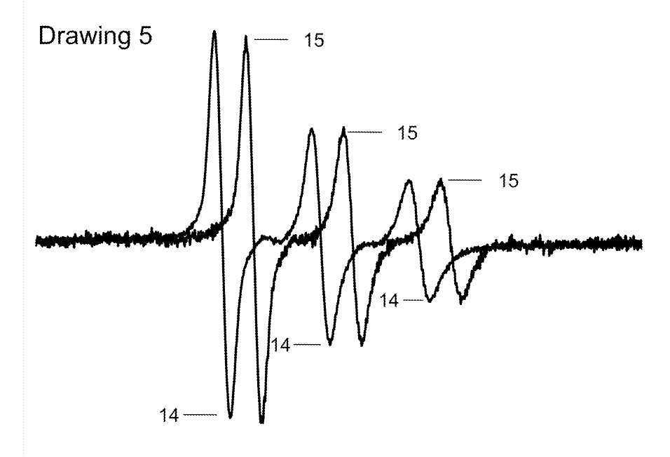

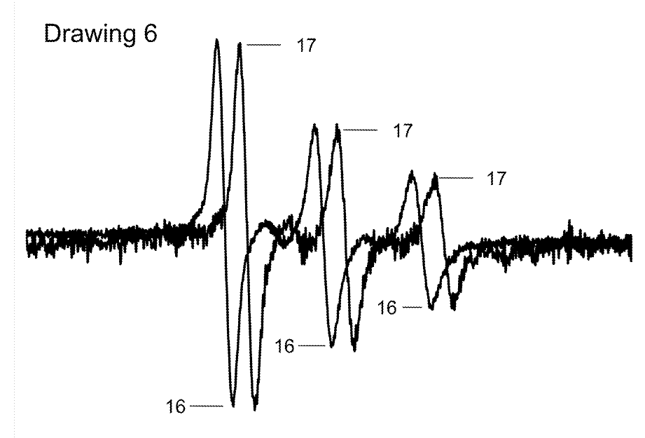

Drawing 1 of 6 demonstrates detection of protein interactions despite considerable mobility of the labeled site. Spectrum 1 is the X-band EMR spectrum of spin labeled phosphorylase B (PB). Though PB is known to have a molecular weight of approximately 200 kDa, spectrum 1 is consistent with the mobility of a much smaller molecule. The labeled site's angular mobility is over 100 times that of the protein itself as an equivalent solid body. Following treatment that promotes dimerization of PB into phosphorylase A (PA), spectrum 2 was recorded. Of note is the new lineshape feature indicated by the number, 3. This feature, consistent with the slower angular mobility of a larger molecule, demonstrates the partial conversion of the PB population into PA. Distribution between the two populations is shown in FIG. 2 of 6.

Spectra 4 and 5 in Drawing 2 of 6 are the leftmost 40% of spectra 2 and 1 in Drawing 1 of 6 respectively. Spectrum 4 includes contributions from both PA and PB while spectrum 5 is wholly that of PB. Thus the spectrum of PA may be isolated via digital titration (titration endpoint determined by isolation of non-PB structures such as that noted by the number 7). Spectrum 6 is the EMR spectrum of PA, derived by the following operation: Spectrum 4—0.67×Spectrum 5 (PA/PB distribution was confirmed by analytical ultracentrifugation (data not shown)). Thus this invention not only detects the protein interaction per se but also differentiates the labeled protein in its “free” and “bound” forms. The free/bound composite spectrum is amenable to quantitative analysis and the interaction essentials readily characterized. For the case presented, the extent of the PB to PA conversion is determined whereas, for most protein interactions, comparable analyses leads to reliable determination of association/dissociation constants.

Drawing 3 of 6 demonstrates detection and characterization of a “weak” protein interaction. Spectrum 8 is the X-band EMR spectrum of labeled bovine trypsin. Spectra 9 and 10 are those of labeled bovine trypsin plus bovine serum albumin (BSA) equimolar with trypsin and in ten-fold excess respectively. The “weakness” of the interaction is reflected in the relatively small diminution in height of the peaks, even in the presence of excess BSA. Even so, the distribution of labeled trypsin in free form is readily assessed and association/dissociation constants determined. During the time frame of data collection, trypsin actively digests BSA (data not shown). Thus the trypsin/BSA interaction is detected and characterized even as the binding partner is being degraded. Modulation of this interaction is demonstrated in the next drawing.

Drawing 4 of 6 repeats two spectra from Drawing 3 of 6. Spectra labeled 11 and 12 are those for labeled bovine trypsin and labeled trypsin plus ten-fold molar excess BSA respectively. Spectrum 13 is that for conditions identical to those for spectrum 12 with the addition of bovine trypsin inhibitor equimolar with labeled trypsin. The fact of binding inhibition is clear in the near equivalence of spectra 11 and 13. These data also support the presumed mechanism of protease inhibition by the inhibitor: inhibition of protease binding to the substrate.

Drawings 5 of 6 and 6 of 6 are closely related. Spectrum 14 in Drawing 5 of 6 is the W-band EMR spectrum for labeled bovine trypsin. Spectrum 15 is that for labeled trypsin in the presence of excess trypsin inhibitor. The binding of trypsin inhibitor, a very small protein, to trypsin creates no discernable change in angular mobility as expressed in near-equivalence of the two lineshapes. However, the shift in the spectrum does demonstrate the invention's ability to discern changes in polarity at the labeled site as a detector for protein interactions. In this instance, the upfield direction of the shift is consistent with an increase in polarity at the labeled site when trypsin inhibitor binds to trypsin. This observation is repeated in the case of trypsin inhibitor binding to a second protease, chymotrypsin, as shown in Drawing 6 of 6. Spectrum 16 is that for labeled chymotrypsin. Binding of trypsin inhibitor to chymotrypsin produces an upfield shift, analogous to that for inhibitor binding to trypsin.

These data taken collectively demonstrate the generality and breadth of the invention. Protein interactions are determined without media component separation: the invention is amenable to detection and characterization of covalent protein interactions and interactions that exist in dynamic equilibrium, regardless of whether the interaction is electrostatic or lipophilic in nature. Protein interactions are detectable and quantifiable regardless of the relative mobility of the labeled site: there are no a priori constraints on label site selection and multiple, sequential sites may be labeled for confirmation and insurance of bio-activity. “Weak” interactions are also detectable as are interactions formed concurrent with active degradation of one of the interacting entities: this invention removes qualifiers associated with many extant methods. Protein interactions are detectable that induce changes in angular mobility and/or polarity of the labeled site: this invention naturally accommodates protein interactions with but not limited to interactions with other proteins, polynucleotides and extended structures such as cell membranes and intact cells. Protein interaction modulation is readily observed in comparative analyses regardless of the source of the modulation: this invention allows determination of protein interaction dependence on bio-relevant variations in, e.g., temperature, pH and ionic strength as well as examination of chemical, biochemical and biological modulators. Protein interaction detection and characterization is permitted in media of any desired complexity: near physiological conditions can be maintained and bio-relevant distribution of both protein interaction participants and modulators may be established.

Test materials, whether natural, expressed or synthetic, may be obtained from any and all sources.

The term “Protein” as used herein encompasses all naturally occurring proteins, modified naturally occurring proteins, protein fragments, protein degradation products, expressed and/or synthetic proteins, and polypeptides.

The term “Protein Interaction” is used in the most general sense as an event or events in which a protein associates with one or more molecules, complexes or structures by any process or mechanism.

The term “Protein Interaction Modulation” is used in its most general sense as any change in protein interaction relative to the non-modulated results. Modulation may include but not be limited to qualitative changes in the data or quantitative changes in reaction and equilibrium kinetics, reaction mechanism determination, association—dissociation constants, and stoichiometric dependences.

The term “Protein Interaction Modulator” is used in its most general sense as any entity, change in test materials, or change in test conditions that effects a change in protein interaction. Modulators may include but not be limited to changes in temperature, acidity (basicity), or the introduction of one or more chemical, biochemical or biological entities. Exemplar of a “Modulator” is any chemical, biochemical or biological entity in use or intended for use as a pharmaceutical agent.

The term “Spin Labeling” is the attachment of a paramagnetic entity, exemplars of which include, but not limited to, nitroxide-bearing molecules.

“Angular mobility” is construed as the angular displacement in time of the observed protein as a solid-body equivalent combined with local, angular displacement events. In other contexts “angular mobility” is the composite of rotational, segmental and local angular displacement phenomena.

Data generated via these methods encompasses the qualitative observation of protein interactions and interaction modulation per se, and the quantitative determination of, but not limited to, reaction and equilibrium kinetics, reaction mechanism determination, association/dissociation constants, and stoichiometric dependences.

In broad embodiment, the present invention is a set of related methods for detection and characterization of protein interactions, protein interaction modulation, and protein interaction modulators via assessment of differential angular mobility and/or differential polarity of protein interaction reactants and products.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

We claim:

1. Detection and characterization of the interaction of proteins with other biological entities such as proteins, polynucleotides, and cell membranes via the combined utility of Electron Magnetic Resonance spectroscopy (EMR, also known as Electron Spin or Electron Paramagnetic Resonance spectroscopy) and Spin Labeling (SL).

2. Detection and characterization of modulation of the interaction of proteins with other biological entities such as proteins, polynucleotides, and cell membranes via the combined utility of Electron Magnetic Resonance spectroscopy (EMR, also known as Electron Spin or Electron Paramagnetic Resonance spectroscopy) and Spin Labeling (SL).

3. Detection, identification and characterization of modulators of the interaction of proteins with other biological entities such as proteins, polynucleotides, and cell membranes via the combined utility of Electron Magnetic Resonance spectroscopy (EMR, also known as Electron Spin or Electron Paramagnetic Resonance spectroscopy) and Spin Labeling (SL).
